(19) United States
(12) Patent Application Publication
(10) Pub. No.: US 2023/0151514 A1
(43) Pub. Date: May 18, 2023

(54) HYSTERESIS IN TEXTILE SENSOR

(71) Applicant: Footfalls and Heartbeats (UK) Limited, Nottingham,Nottinghamahire (GB)

(72) Inventors: Cristina ISAIA, Nottingham, Nottinghamshire (GB); Byron Kirk SALISBURY, Nottingham, Nottinghamshire (GB); Andrew Glyn THOMPSON, Nottingham, Nottinghamshire (GB); Simon Adair MCMASTER, Nottingham, Nottinghamshire (GB)

(21) Appl. No.: 17/912,808

(22) PCT Filed: Mar. 19, 2021
(86) PCT No.: PCT/GB2021/050695

§ 371 Date: Sep. 19, 2022

(30) Foreign Application Priority Data
Mar. 19, 2020 (GB) 2004024.2

(51) Int. Cl. D04B 1/12; A61B 5/00

(52) U.S. Cl. CPC D04B 1/12; A61B 5/6804; D10B 2403/02431; D10B 2401/16

(57) ABSTRACT

According to an aspect of the present invention there is provided a knitted textile. The knitted textile comprises a knitted textile sensor. The knitted textile sensor comprises an electrically conductive yarn and a plurality of stitches that form a defined sensing stitch pattern. The sensing stitch pattern comprises stitches selected from the group consisting of: jersey stitches; tuck stitches; and/or miss stitches; as well as any combination thereof. The sensing stitch pattern provides a measurable contact resistance that varies with a force applied to the textile. The knitted textile comprises a knitted support structure within which the knitted textile sensor is integrated. The knitted support structure comprises a plurality of stitches that form a defined support stitch pattern, the support stitch pattern comprising stitches selected from the group consisting of: jersey stitches; tuck stitches; and/or miss stitches; as well as any combination thereof. The support stitch pattern has a smaller percentage of tuck stitches than the sensing stitch pattern. The textile is configured to elongate and retract in the wale direction in a repeatable manner, even after multiple washing cycles. Also provided are a garment comprising the knitted textile structure and knitted textile sensor.

### TECHNICAL FIELD

The present disclosure relates to a textile based sensor device, and to a textile and a garment incorporating the textile sensor. The disclosure further relates to sensors that are incorporated into textiles, such as medical support bandages, that are suitable for adequately measuring repeated movements.

### BACKGROUND

An increasingly important area in textile design is that of “intelligent textiles” in which electrical signals representing physiological data are collected from garments and transmitted to remote locations for the purpose of, for example, monitoring, assessment, and intervention by health care professionals. Often, such textile devices are generally not truly intelligent textiles, as they comprise solid-state electronics placed in a textile shell and worn as apparel. As a result, such devices are often bulky or incongruous and can impair normal movement.

Truly intelligent textiles are in development in which the sensor is embedded within and forms part of the textile. In other words, the textile itself is the sensor. Examples of intelligent textile sensors of this kind are found in published patents and applications of the applicant, such as international patent applications WO 2014/122619 and WO 2017/037479.

Textile sensors, and particularly knitted textile sensors, are useful and more in-demand than ever before. Unfortunately, their performance can be susceptible to effects experienced by all textiles, particularly knitted textiles. One of these effects is that cycles of elongation of textiles is not repeatable. The hysteresis curve exhibited by a textile under elongation differs between cycles in an unpredictable manner. Simply put this means that when stretched in any direction or combination of directions it is very unlikely that the textile will return to exactly the same state (e.g. shape and configuration) before any forces were applied. Knitted textiles are designed to fit over almost any organic shape and return to a relaxed state when removed. This relaxed state may look identical to the “eye”, however at the yarn level it is almost never the same. In addition the process that returns the garment to the relaxed state will never follow the same “path of relaxation” to that state.

Accordingly, when this effect is seen in textile sensors, it introduces a degree of uncertainty over accuracy, linearity, and repeatability of the output signal from the sensor. Such an effect prevents the textile returning to a ‘zero’ state which can impair the capacity to act as a reliable sensor. In turn, this significantly limits the utility of textile sensors in range of motion sensor applications, and instead restricts them to uses as counters or as on-off, binary sensors. Anything beyond these motion sensors requires repeated, time-consuming calibration, and even then the calibration cannot be considered to be entirely accurate.

It is an aim of the present invention to address one or more of the disadvantages associated with the prior art.

### SUMMARY OF THE INVENTION

According to an aspect of the present invention there is provided a knitted textile. The knitted textile comprises a knitted textile sensor. The knitted textile sensor comprises an electrically conductive yarn and a plurality of stitches that form a defined sensing stitch pattern. The sensing stitch pattern comprises stitches selected from the group consisting of: jersey stitches; tuck stitches; and/or miss stitches; as well as any combination thereof. The sensing stitch pattern provides a measurable contact resistance that varies with a force applied to the textile. The knitted textile comprises a knitted support structure within which the knitted textile sensor is integrated. The knitted support structure comprises a plurality of stitches that form a defined support stitch pattern, the support stitch pattern comprising stitches selected from the group consisting of: jersey stitches; tuck stitches; and/or miss stitches; as well as any combination thereof. The support stitch pattern has a smaller percentage of tuck stitches than the sensing stitch pattern. The textile is configured to elongate and retract in the wale direction in a repeatable manner.

By elongation and retraction in a repeatable manner, it is meant that the textile follows a hysteresis curve during elongation and retraction of the textile that is identical or substantially identical during a plurality of consecutive cycles and which action can be repeated in different tests (i.e. the similarity is not a random event). Thus, the textile becomes a viable sensor for use in measurement of elongation and retraction of, for example, joint extensions. By creating a knitted textile sensor comprising a stitch pattern whose contact resistance varies with force, the textile's motion is measurable and, by virtue of its repeatability, capable of correlation with the movement of the joint it is configured to measure.

The support structure is important for maintaining the sensor in position in general, for maintaining its shape when elongated and retracted, and for permitting the sensor to be used in different situations. By incorporating more tuck stiches in the sensing stitch pattern, the support structure is able to stretch with the sensor during elongation and retraction, meaning that the support structure does not restrict the movement of the sensor.

The support stitch pattern may comprises stitches selected from the group consisting of jersey stitches and miss stitches. Miss stitches reduces creasing in the textile. Creases may hamper the smooth elongation and retraction of the textile sensor.

The support stitch pattern may include one or more double miss stitch arrangements. A double miss stitch arrangement comprises two consecutive miss stitches in the same wale. Double miss stitch arrangements are particularly effective at reducing creasing.

Optionally, the knitted textile comprises a boundary zone contiguously interconnecting the courses of the knitted textile sensor and of the knitted support structure, wherein the boundary zone is configured to maintain the position and aspect ratio of the knitted textile sensor relative to the knitted support structure. By contiguously interconnecting, it is meant that there is a continuous connection along the courses, rather than a seam or break that would otherwise cause unknown movements. In some embodiments, the boundary zone is part of the knitted textile sensor and part of the knitted support structure. In other embodiments, the boundary zone comprises a different yarn. Maintaining the aspect ratio and position of the sensor further enhances the repeatable hysteresis curve exhibited by the sensor.

The boundary zone may comprise a plurality of stitches that form a defined boundary stitch pattern, wherein the boundary stitch pattern comprises stitches selected from the group consisting of: jersey stitches; tuck stitches; and/or miss stitches; as well as any combination thereof. The boundary stitch pattern may have a higher percentage of tuck stitches than the support stitch pattern. The boundary stitch pattern may have a higher percentage of tuck stitches than the sensing stitch pattern. The boundary zone may comprise stitches selected from the group consisting of jersey stitches and tuck stitches. Creating a boundary zone that is stiffer than the sensor is achieved by incorporating more tuck stitches. This improves the repeatability of the sensor by putting limits on its elongation, without reducing its efficacy as a sensor.

The boundary zone may be at least two wales wide. The tuck stitches in the boundary zone may be staggered. By staggered, it is meant that in tuck stitches are provided in alternate wales between courses, or in an alternating pattern along the wales. So, for example, where the boundary zone is two wales wide, staggered tuck stitches lead to the first tuck stitch being in the first wale in the first course, and the second tuck stitch being in the second wale and the second course, and then back to the first wale in the next course and so on.

Optionally, the knitted textile comprises a stabilising structure attached to one side of the knitted textile structure, wherein the stabilising structure comprises an elastic fabric. By side, the surface of the textile is intended, although a stabilising structure may alternatively be attached to one or more edges of the textile. Attaching a stabilising structure to one surface of the textile adds a further limit and stiffness to the sensor, as well as providing insulation for the sensor from other surfaces.

The stabilising structure may comprise an open fabric configured to enable heat dissipation therethrough. This is particularly useful for uses where the textile is to be applied to skin as it reduces the likelihood of heat build-up affecting the effectiveness of the sensor.

Optionally, 50% of the stitches in the sensing stitch pattern are jersey stitches, and the remaining 50% of stitches comprise a combination of miss stitches and tuck stitches. In some embodiments, more than 25% of the stitches in the sensing stitch pattern are tuck stitches with the remainder of the stitches in the sensing stitch pattern being miss stitches. Such a stitch pattern is particularly effective in showing repeatable characteristics.

The electrically conductive yarn may comprise a multifilament yarn. Multifilament yarns are advantageous as they improve the repeatability of the textile, further improving its use in sensing elongation and retraction.

The structure may be arranged to form a sleeve. A sleeve structure is highly useful in reducing waisting, creasing, in maintaining the aspect ratio of the textile, and for use on limbs.

According to another aspect of the invention, there is provided a garment comprising the knitted textile structure described above. The garment may comprise a sleeve.

According to another aspect of the invention, there is provided a knitted textile sensor comprising: a support region comprising yarn and a plurality of stitches that form a defined support stitch pattern wherein the support stitch pattern comprises stitches selected from the group consisting of: jersey stitches; tuck stitches; and/or miss stitches; and wherein at least 50% of the stitches in the support stitch pattern are jersey stitches; and a sensor region comprising an electrically conductive yarn and a plurality of stitches that form a defined sensing stitch pattern, wherein the sensing stitch pattern comprises stitches selected from the group consisting of: jersey stitches; tuck stitches; and/or miss stitches; wherein 50% of the stitches in the sensing stitch pattern are jersey stitches, at least 25% of the stitches in the stitch pattern are tuck stitches, and the remainder of the stitches in the sensing stitch pattern are miss stitches; and wherein the stiffness of the sensing stitch pattern is higher than the stiffness of the support stitch pattern.

According to another aspect of the present invention there is provided a knitted textile. The knitted textile comprises a knitted textile sensor. The knitted textile sensor comprises an electrically conductive yarn and a plurality of stitches that form a defined sensing stitch pattern. The sensing stitch pattern comprises stitches selected from the group consisting of: jersey stitches; tuck stitches; and/or miss stitches; as well as any combination thereof. The sensing stitch pattern provides a measurable contact resistance that varies with a force applied to the textile. The knitted textile comprises a knitted support structure within which the knitted textile sensor is integrated. The textile is configured to elongate and retract in the wale direction in a repeatable manner.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

One or more embodiments of the invention will now be described, by way of example only, with reference to the accompanying drawings, in which:

FIG. 1A is a diagrammatic view of two interconnected yarn units in a single jersey knit stitch pattern;

FIG. 1B is a diagrammatic view of a plain single jersey knit stitch pattern for use in a textile sensor;

FIG. 2 is a diagrammatic view of an alternative embodiment of the textile sensor which has a knit stitch pattern having single jersey stitches, miss stitches, and tuck stitches;

FIG. 3A is an electron microscope photograph of a fabric sample comprising a multi-filament, twisted polyester yarn with a conductive coating (silver) which is knit in a plain single jersey stitch pattern in an un-deformed state;

FIG. 3B is an electron microscope photograph of a fabric sample comprising a stainless steel staple fibre spun yarn knit in a plain single jersey stitch pattern in an un-deformed state;

FIG. 4 is a schematic representation of a textile according to an embodiment of the invention;

FIG. 5 is a stitch pattern for use in the textile of FIG. 4;

FIG. 6 is a stitch pattern for use in the textile of FIG. 4;

FIG. 7 is a stitch pattern for a support fabric for use in the textile of FIG. 4;

FIG. 8 is a schematic perspective representation of a textile according to a further embodiment of the invention;

FIG. 9 is a schematic perspective representation of a textile according to another embodiment of the invention;

FIG. 10 is an experimental setup for testing hysteresis of textile samples;

FIG. 11 is an example textile sample for use in the experimental setup of FIG. 10;

FIGS. 12A to 12C are graphs showing the hysteresis of a first textile sample during testing using the experimental setup of FIG. 10;

FIGS. 13A to 13C are graphs showing the hysteresis of a second textile sample during testing using the experimental setup of FIG. 10;

FIGS. 14A to 14C are graphs showing the hysteresis of a third textile sample during testing using the experimental setup of FIG. 10;

FIGS. 15A to 15C are graphs showing the hysteresis of a fourth textile sample during testing using the experimental setup of FIG. 10;

FIGS. 16A to 16F show testing of knee and elbow sleeves using a HUMAC NORM machine, where the figures individually show: a) close-up of the knitted sensor in the knee sleeve; b) knee sleeve in flexion; c) knee sleeve in extension; d) experimental set-up; e) elbow sleeve in flexion; f) elbow sleeve in extension;

FIGS. 17A and 17B show graphs of: a) fabric resistance variation with time for the E5 elbow sleeve during 1st-200th cycles and 800th-1000th cycles and b) hysteresis curves for the corresponding interval; and

FIGS. 18A and 18B show radar chart performance comparisons for the a) elbow and b) knee sleeves.

FIGS. 19A to 19C show graphs of the electrical response of a sensorised sleeve during 50 flexion-extension cycles for a) a new sample, b) a 50-time washed sample and c) a 100-time washed sample.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

Prior to setting forth the invention, a number of definitions are provided that will assist in the understanding of the invention.

As used in this description, the singular forms “a,” “an,” and “the” include plural referents unless the context clearly dictates otherwise. Thus, for example, the term “a sensor” is intended to mean a single sensor or more than one sensor or to an array of sensors. For the purposes of this specification, terms such as “forward,” “rearward,” “front,” “back,” “right,” “left,” “upwardly,” “downwardly,” and the like are words of convenience and are not to be construed as limiting terms. Additionally, any reference referred to as being “incorporated herein” is to be understood as being incorporated in its entirety.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

As used herein, the terms “distal” and “proximal” are used to refer to orientation along the longitudinal axis of the apparatus. Since the fibres of the invention are elongate in nature and conform to a single dimension, in use the distal direction refers to the terminus of the fibre furthest away from the source or receiver and the proximal direction to the terminus of the fibre closest to the source or receiver. It should be noted that the term proximal should not be confused with the term ‘proximate’, which adopts its conventional meaning of ‘near to’.

The phrase “skin surface” as used herein is intended to refer to the epidermal surface of a subject, typically a human or animal, that is being monitored. In mammals, the skin comprises the outer epidermal layer and the underlying dermis, as well as and supporting tissues including the vasculature associated with the skin.

For purposes herein, a “motion artefact” is any error in the perception or representation of a signal introduced by motion of a sensor device or a subject to which the device is applied. Motion may be caused by voluntary or involuntary movements, such as locomotion, of the subject wearing the device of the invention.

As used herein, the term “contact resistance” is used to refer to the total electrical resistance of a portion of the textile due to contacting yarns. The contact resistance varies with the yarn contact area and can change based upon the applied force, weight or tension applied to the textile. The equation

$${R}_{c} = \frac{ρ}{2} \sqrt{\frac{π H}{F}}$$

is a representation of the Holm contact resistance equation, where R_{c} is contact resistance, p is material resistivity, H is material hardness, and F is the normal force. The equation

$${R}_{c} = \frac{ρ}{2} \sqrt{\frac{π H}{n P}}$$

is another representation of the Holm equation, which is more relevant to textile-based contact resistance. F is replaced by nP, where n is the number of contact points between adjacent yarn in the textile, and P is the contact pressure. Material hardness and electrical resistivity are constants that depend on the material properties of a textile. Contact resistance is therefore inversely proportional to the number of contact points and the contact pressure. That is, more contact points result in lower contact resistance. Therefore, as the number of contact points and/or contact pressure increases, contact resistance decreases. As used herein, contact resistance provides a measure of electrical conductivity in a yarn or textile. At the “micro” scale, surface roughness limits surface-to-surface contact. In addition, as pressure increases, the number of contact points increases, and eventually at the “nano” scale individual contact points “combine” into a larger contact area. “Integration as Summation” and the “Finite Element Method (FEM)” are techniques that can be used to determine the limits of these contacts points and therefore the contact area they produce.

As used herein, the term “length-led resistance” or “length-related resistance” is used to refer to the total electrical resistance of a portion of the textile due to length variation of the conductive yarn. The length-related resistance varies with yarn length and can change based upon applied force, weight, or tension applied to the textile. The equation

$$R = \frac{ρ L}{A}$$

is representation Ohm's law, which is relevant to length-related resistance, where R is length-related resistance, p is resistivity, L is length of yarn, and A is cross-sectional area of yarn. The sensing mechanism may also be based on the change in the conduction path due to transformation of the equivalent electrical network associated to the fabrics structure.

As used herein, the term “textile” and “fabric” refers to a flexible material manufactured from a plurality of individual fibres that have been combined. A textile or fabric may be woven, knitted, crocheted, spread or made by any other kind of interlacing that may be achieved using fibres. A “fibre” used in relation to a textile refers to any substantially elongate yarn or thread.

For the purposes of this application, a “multifilament yarn” is defined as a yarn formed of a plurality of fine continuous filaments grouped together. The filaments are generally continuous in length along the length of the yarn, so that each filament can be considered to extend along the length of the yarn. Multifilament yarns may comprise a twist in the yarn to facilitate handling.

As used herein, the term “staple fibre yarn” is defined as yarn formed of staple fibres, each having a discrete staple length. Many staple fibres are spun together to form a length of yarn, with the length of the yarn being much greater than the length of any individual staple fibre.

As used herein, a “miss stitch” is defined as a knitting stitch in which at least one needle holds the old loop and does not receive any new yarn across one or more wales. A miss stitch connects two loops of the same course that are not in adjacent wales.

For purposes herein, “plain stitch” refers to a knitting stitch in which a yarn loop is pulled to the technical back of a fabric. A plain stitch produces a series of wales or lengthwise ribs on the face of the fabric and courses, or cross-wise loops, on the back. A plain stitch can also be referred to as a “single-knit jersey stitch” or a “single jersey stitch.”

A “tuck stitch” is defined for use herein as a knitting stitch in which a yarn is held in the hook of a needle and does not form a new loop. Tuck stitches are typically created by knitting stitches from the current course together with the same stitches one or more courses below.

The term “repeatability” is defined for use herein as a consistent exhibition of particular physical characteristics, specifically a measurable contact resistance change and/or a length-led resistance change within the textile sensor, over a series of consecutive cycles of tensile elongation and relaxation. Substantial repeatability may be measured as being obtained over at least 2 cycles, over at least 5 cycles, over at least 10 cycles, over at least 25 cycles, over at least 50, and/or over at least 100 cycles. Repeatability over even a few cycles of elongation and relaxation in knitted textiles is not a trivial matter as knitted textiles are typically unreliable in exhibiting hysteresis.

Percentage Permanent Stretch (PPS) is defined as a measure of the stretch and recovery of a fabric when subjected to a cyclical load.

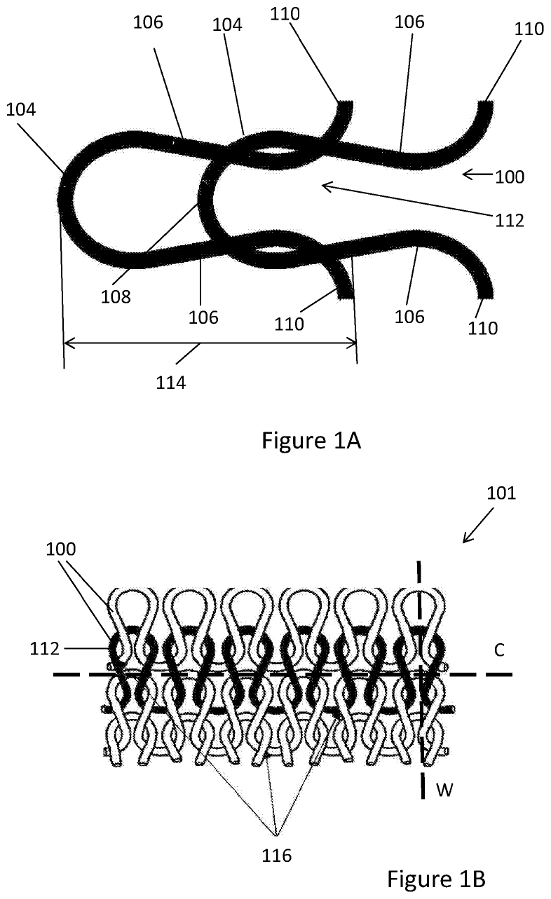

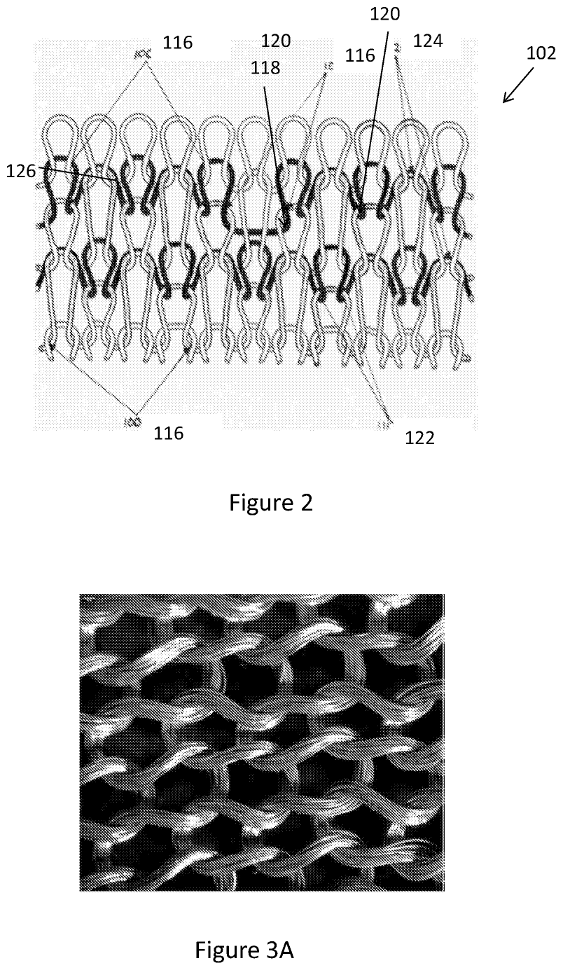

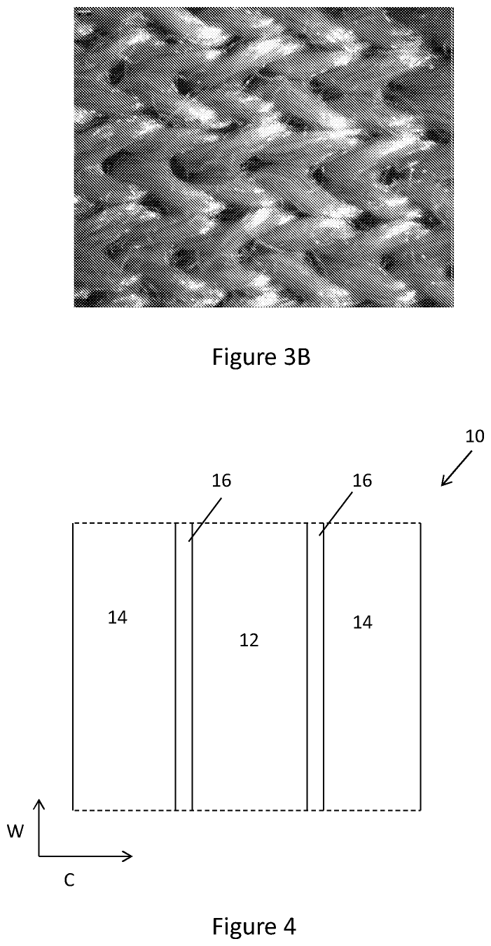

To help set the invention, the above terms relating to stitches are put into context through the schematic representations of FIGS. 1A, 1B, and 2, and the above terms relating to yarn types are explained using FIGS. 3A and 3B.

FIG. 1A is a schematic representation of a textile, which may also be called a jersey knit textile, comprising a single jersey stitch 100 and illustrates the concept of yarn contact area. In a jersey knit textile, a needle loop 104, or yarn unit, comprises a head 104 and two side legs 106 that form a noose 108. At the base of each leg 106 is a foot 110, which meshes through the head 104 of a sinker loop 112 formed at the previous knitting cycle. The leg 106 of the needle loop 104 passes from one side (or face) to the other side/face of the sinker loop 112 across the leg 106 and head 104 of the sinker loop 112, and then loops around to pass back across the head 104 and opposite leg 106 of the sinker loop 112 to back to the original side/face of the sinker loop 112. Stitch length 114 is defined as a length of yarn which includes the needle loop 104 and a half of the sinker loop 112 on either side of it.

FIG. 1B is a schematic drawing of a single jersey stitch pattern 101. In this pattern, interconnecting stitch loops touch at single jersey contact points 116. In a single jersey stitch pattern, one stitch contacts an adjacent stitch essentially on only one side, or surface, of the adjacent stitch (or fabric) at a time. That is, in two interconnected stitch loops, the legs of a first stitch loop contact the feet of a second, adjacent stitch loop on one surface of the second stitch loop. On the opposite surface of the second stitch loop, the head of the first stitch loop contacts the legs of the second stitch loop. As a result, single jersey contact points are limited to relatively small crossover points of adjacent loops.

The yarns are arranged into courses, and the loops interconnect to form wales that extend perpendicularly to the courses. In FIG. 1B, an example course is indicated with a dotted line labelled ‘C’, while an example wale is indicated with a dotted line labelled ‘W’. The yarns in each course interact with yarns in the immediately adjacent yarns. Yarn contact area, i.e. the size of the regions of contact between the adjacent yarns, varies depending upon the movement of the textile and forces upon the textile.

Yarn contact area is influenced by many different variables of the textile, and has a direct influence on contact resistance of a textile formed of electrically conductive yarns. Contact resistance of a textile is measurable to determine various parameters. Contact resistance is associated with the conduction characteristic of the yarn contact surface area. A larger yarn contact area and less surface roughness of the yarn surface results in a lower resistance to electrical signals travelling through the textile. Thus, an increase in yarn contact area causes a proportional decrease in contact resistance. Yarn variables, stitch variables, and textile variables each influence yarn contact area, and thereby provide variables that can be used to specifically design a textile having a yarn contact area, and thus contact resistance, adapted for a particular sensing activity or use.

Variables that can affect contact resistance include: yarn type or composition (e.g. filament or staple fibre yarn); yarn fabrication method; yarn count; stitch type, composition, or pattern; stitch length; stitch percentage; mean electrical resistivity (MER); fabric thickness; fabric weight; optical porosity (OP); and percentage permanent stretch (PPS).

FIG. 2 is a schematic drawing of a stitch pattern 102 having single jersey stitches as well as miss stitches and tuck stitches. The stitch pattern 102 having miss and tuck stitches includes single jersey contact points 116, as well as additional contact points at the miss stitches 118 and tuck stitches 120. A tuck stitch contact point 122 occurs when a tuck stitch loop interconnects in a course with adjoining stitch types. A tuck loop contact point 124 occurs when the tuck loop of a tuck stitch presses upon the held loop of a tuck stitch. A held loop contact point 126 is formed when the held loop of a tuck stitch is forced against an adjacent stitch loop.

As compared to the plain single jersey stitch pattern 100 seen in FIG. 1B, the different contact points and areas shown in the tuck stitch and miss stitch structures in FIG. 2 allow for different yarn contact areas between textiles having different stitch patterns, and therefore contact resistance that can be designed specifically for a given application or sensing activity and correlated with a desired measurement parameter.

The textile structures of the type shown in FIG. 2 are the subject of patent application numbers PCT/IB2014/058866 and PCT/IB2014/063929. In line with stitch patterns described in these applications, the textile structure in FIG. 2 comprises a stitch pattern having 50% jersey stitches, with the remaining 50% having a combination of tuck stitches and miss stitches. These percentages are derivable from FIG. 2 by counting the stitches: every other stitch is a jersey stitch, with tuck and miss stitches located therebetween.

FIG. 3A shows a photograph of a textile formed of single jersey stitches using a multifilament yarn. For contrast, FIG. 3B shows a photograph of a textile formed of single jersey stitches using a staple fibre yarn. The multifilament yarn of FIG. 2A has filaments that are maintained tightly grouped within the yarn structure, without straying from the stitch pattern. The filaments may follow different paths through the yarn, but generally are all contained within a tight bundle. In contrast, the staple fibres of the staple fibre yarn of FIG. 3B are mostly contained within the stitch pattern but some of the staple fibres, are seen to stray and extend outwardly.

Having defined and illustrated the stitch and yarn types to be discussed herein, FIG. 4 illustrates a portion of a textile 10 according to an embodiment of the invention. The textile 10 includes a knitted textile sensor 12. The wale, W, and course, C, directions of the knitted yarns in the textile 10 are indicated; as is convention, the wale direction is in the vertical direction of the page, while the course direction is horizontally across the page. The textile sensor 12 is provided within a support fabric 14, which may also be referred to as a support structure and which is typically also knitted textile. The support fabric 14 is positioned along the sides of the textile sensor 12 at least. In other embodiments, the support fabric 14 may fully or partially surround the textile sensor 12, or extend along other edges of the textile sensor 12 such as along its upper and lower boundaries.

The support fabric 14 and textile sensor 12 are interconnected via a boundary zone or zones 16 that covers at least the overlap of the support fabric 14 and textile sensor 12 along its edges. The boundary zone 16 is at least one stitch (wale) wide. It will be appreciated that although the boundary zone 16 is depicted as separate to the support fabric 14 and textile sensor 12, the boundary zone 16 is formed of overlaid and overlapping ends of threads from the support fabric 14 and textile sensor 12. Hence, the boundary zone is contiguous with the support fabric 14 and the sensor 12.

The textile 10 of FIG. 4 is provided by way of example only. In embodiments, a plurality of textile sensors may be incorporated into a support fabric. In some embodiments, more than one support fabric may be used. Furthermore, one or more electrical connections may be provided to connect the textile sensor to a sensing unit for measuring contact resistance in the textile. The electrical connection may comprise one or more of: an electrically conductive yarn or yarns laid into the support fabric; a knitted electrical pathway comprising the electrically conductive yarn arranged into a suitable stitch pattern and knitted between the support fabric; a connection created using conductive ink printed onto the support fabric; or by a conventional wired connection.

Returning to the embodiment of the textile provided in FIG. 4, the textile sensor is configured for and is capable of detecting elongation and movement, such as flexion, of the textile 10 in the wale direction W, and the textile 10 itself is configured to enhance the capabilities of the textile sensor 12. The textile sensor 12 is therefore effectively acting as a proxy sensor for strain detection. Strain and/or elongation detection may be used in medical applications for monitoring joint movements (e.g. knee and elbow) during physical rehabilitation, particularly where patients are required to repeat movements a set number of times or where full extension of a joint is desirable. The textile may also be used to determine elongation in physical structures, and may be incorporated into or placed over vibrating or moving parts.

Conventionally, knitted textile sensors have been considered inaccurate for determining elongation or movement due to the inherently unpredictable movement of textiles subjected to force. The structure of these conventional knitted textiles changes with elongation in a manner that is not repeatable; the movement of the yarns in the stitches relative to one another, the movement of the fibres in the yarns relative to one another, and the friction therebetween change with each movement. Accordingly, when conventional knitted textiles are subjected to the stress of extension, they do not automatically return to the same configuration when force is removed. The textile may be deformed or elongated following each cycle of extension and relaxation. The phenomenon of deformation and subsequent relaxation in knitted textiles is referred to as ‘hysteresis’. Hysteresis is generally defined as the lag in response of a system to forces placed upon it, and in a knitted textile sensor hysteresis is exhibited during elongation and relaxation of the textile. Hysteresis curves generated for conventional knitted textiles are typically erratic, with different extensions observed with each cycle of extension and relaxation. For the avoidance of any doubt, cycles referred to herein are cycles of application and release of substantially equal forces to the textile.

It is this characteristic of knitted textiles that means that before the present disclosure they have not been capable of acting as true strain gauges, and, until now, have been unable to adequately mimic conventional strain gauges. A true strain gauge requires a measurement of the change in length of an object to be compared with the original length of the object. Neither of these values is determinable using a textile sensor alone because a textile sensor comprises a knitted three-dimensional matrix of yarns, each of which are themselves formed of a multiplicity of fibres, with a great many degrees of freedom, as noted above. A traditional textile sensor attempting to mimic a strain gauge would require constant recalibration after each cycle of extension and relaxation, as well as requiring using other measurement techniques to supplement and compensate for deficiencies in the performance of the textile sensor.

In attempting to identify how a textile sensor may be provided that improves upon the variability of traditional knitted textiles, the inventors have designed textiles and textile sensors whose fibres interact and cooperate in a highly repeatable way that enables measurement of movement of the fabric, with contact resistance and/or length-led resistance within the textile sensor used as a proxy for elongation, and therefore strain, in the wale direction. These textiles and sensors benefit from a suite of solutions that when taken alone or in combination provide a hysteresis curve from which movement of the textile can be determined reliably over multiple cycles. In particular, in a particular embodiment it has been determined that maintaining the aspect ratio of the textile sensor during movement is important to providing repeatable and reliable textile sensor output. In a further embodiment, reducing capacity of the textile sensor to stretch/elongate in a non-elastic manner in the wale direction is another important consideration. The solutions that are discussed in turn below aid overcoming in one or both of these problems.

In general, where the textile sensor 12 is referred to in this description, this suitably refers to a sensor of the type described in International patent application numbers PCT/IB2014/058866 and PCT/IB2014/063929. For completeness, this means a knitted textile comprising an electrically conductive yarn arranged into stitches. The stitches form a defined stitch pattern, such as the pattern shown in FIGS. 1B and 2. The stitch pattern comprises jersey stitches, tuck stitches, miss stitches, as well as any combination thereof. The stitch pattern may also comprise laid-in yarns. The stitch pattern provides a measurable contact resistance that varies with elongation of the textile sensor.

When combined with any of the other solutions described herein, the textile sensor itself may be any knitted textile sensor that fulfils the description above. In some examples, the textile sensor comprises 100% jersey stitches.

In some embodiments, for improved repeatability, the sensor has a stitch pattern in which 50% of the stitches are jersey stitches, and the remaining 50% of stitches are a combination of miss stitches and tuck stitches, as exemplified by FIG. 2.

In order to further improve the repeatability of the sensors, the combination of miss and tuck stitches may be tailored to reduce stretch in the wale direction. Wale stretch can be reduced by increasing the number of tuck stitches compared to the number of miss stitches. In other words, the textile sensor has a stitch pattern where 50% of the stitches are jersey stitches and the other 50% of stitches are tuck stitches and miss stitches, with the majority of stitches in the other 50% being tuck stitches. Put in a different way, 50% of the stitches in the stitch pattern are single jersey, more than 25% and less than 50% of the stitches in the sensing stitch pattern are tuck stitches, and the remainder are miss stitches. A reduction in stretch in the wale direction causes the textile to act in such a way that small movements in the textile, i.e. small amounts of strain, will be signalled by a change in resistance. By incorporating more tuck stitches compared to the number of miss stitches, the textile is forced towards its extensible limit. As the textile approaches the limit of extension, the electrical signals will be highly repeatable due to the yarn contact points becoming small and stable. Improving stability in yarn contacts and reducing the size of the contact area leads to reduction in contact resistance and less variability in contact resistance, and thus the signals are more repeatable.

In limiting the stretch of the sensor in the wale direction, its strength and resilience is increased and therefore its repeatability is increased. The sensor may therefore be considered to be ‘stiffer’ or to have a greater tensile ‘strength’ than a pattern having fewer tuck stitches, such as the support fabric as described later. Less elongation causes a fabric to act as a “stiffer” structure, and stiffer structures have low Poisson's ratio. A low Poisson's ratio is desirable to limit movement of the textile to the direction of stretching. This ensures that the change in length is almost directly related to the resistance in the textile.

The stiffness and stretch may be characterised in terms of Poisson's ratio, percent permanent stretch (PPS), or by other means. The design of the stitch pattern, the yarn type, and the knitted process may be tailored to maintain PPS of the sensor in the wale direction within a predetermined range of between 3 and 10%. PPS increases or decreases depending on the percentage of miss stitches and tuck stitches within a stitch pattern. PPS relates to both the course direction and wale direction, and differs for each. The lower the PPS, the lower the contact resistance. PPS is directly proportional to the percentage of either single jersey, miss, and tuck stitches present in the textile. Preferably the PPS value is around 5%. In addition, the PPS of the support fabric can also be tailored so that the combined PPS of a support fabric (details of which are found below) and the sensor is less than the sum of PPSs of the two fabrics individually. Accordingly, by choosing yarn type, stitch pattern, and knitting process carefully, the structure can be designed to have a reduced overall PPS, which is important in ensuring repeatability.

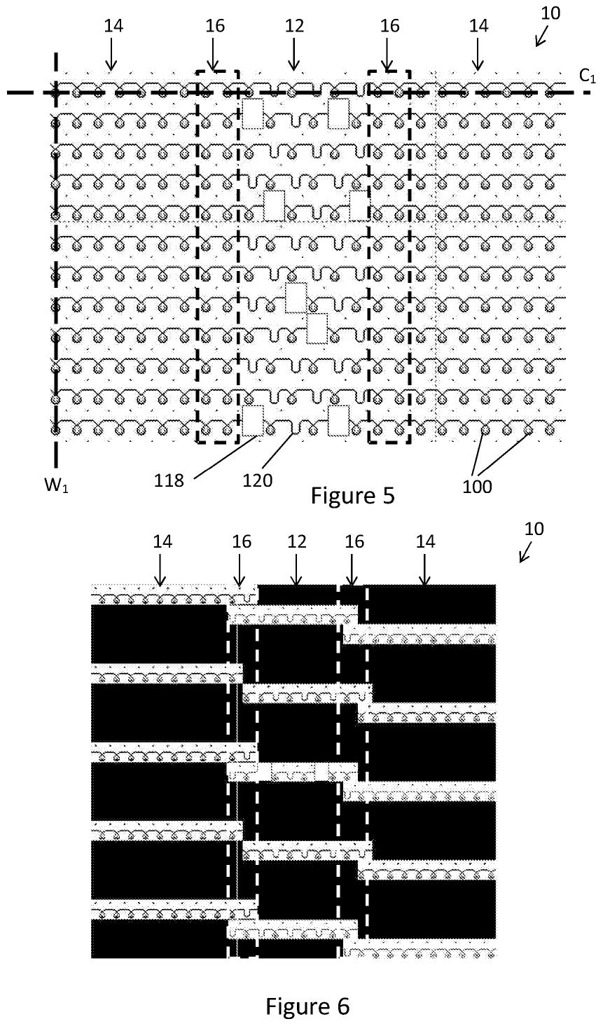

An example of a textile 10 having a stitch pattern 20 of this kind is shown in FIG. 5. In a similar manner to the schematic diagram of FIG. 4, the textile 10 of FIG. 5 has a textile sensor 12 disposed centrally, with a boundary zone 16 demarcated by a dotted box on either side of the sensor 12 and the support fabric 14 on the outer edges. In the schematic depiction of the stitch pattern 20 in FIG. 5, courses extend from left to right (horizontally) across the diagram and wales extend from the top to the bottom (vertically) of the diagram. For the avoidance of doubt as to where the courses and wales lie, the line labelled Ci extends through the uppermost course and the line labelled Wi extends through the leftmost wale.

There are three stitch types depicted in FIG. 5. Single jersey stitches 100 are represented by the looped components. Tuck stitches 120 are represented by U-shaped components. Miss stitches 118 are represented by open boxes.

In the embodiment of FIG. 5, the stitch pattern of the support fabric 14 and boundary zones 16 comprise 100% single jersey stitches. The stitch pattern of the textile sensor 12 comprises 50% single jersey stitches and 50% miss and tuck stitches as described above, the majority of this 50% being tuck stitches—i.e. >25% of the total stitch count for the sensor comprises tuck stitches. In the embodiment shown, there are seventy-two stitches in the textile sensor, and thirty-six of those stitches are single jersey stitches 100. The remainder are tuck and miss stitches 120, 118 in the ratio of 28:8, meaning that approximately 40% of the stitches in this stitch pattern are comprised of tuck stitches 120, while miss stitches 118 make up the remaining 10%. By incorporating this increased ratio of tuck stitches when compared to miss stitches, the embodiment of FIG. 5 has an improved repeatability during cycles of flexion and extension in the wale direction (e.g. during hysteresis), therefore improving its usefulness in detecting movement and elongation of the textile.

While tuck stitches act to reduce deformation and stretching of the sensor in the wale direction and thereby improve repeatability in hysteresis, the miss stitches are also an important component in the textile sensor and, as will be discussed later, can also be usefully incorporated into the support fabric. Miss stitches reduce waisting in the course direction and improve the ability of the stitch pattern to maintain its shape and aspect ratio when under tensile stress during movement. Accordingly, a further improvement is made upon embodiments of the textile sensor having 50% tuck and miss stitches in which tuck stitches are in the majority, by allowing for tuning the relative percentages of tuck and miss stitches to further improve the hysteresis.

The properties of the textile sensor that enable it to be repeatably elongated may be further enhanced by the yarn selection. Embodiments of the textile sensor comprise electrically conductive yarn comprised within the knitted stitch pattern. The yarn plays a role in how the textile sensor reacts to the elongation. Both the interactions between the yarns in each course and the action of the internal fibres of the yarn can be important in defining how the textile sensor behaves.

Accordingly, in particular embodiments of the textile, the textile sensor comprises electrically conductive yarn having a low extensibility. In some examples, the conductive yarn comprises a multifilament yarn. Multifilament yarns are preferable to staple fibre yarn because of their low extensibility properties but also because the ‘noise’ in the measurement raw data is reduced using a multifilament yarn when compared to a staple fibre yarn. Without wishing to be bound by theory, it is believed that this is because multifilament yarns have fewer stray fibres—i.e. are less ‘fuzzy’. Stray fibres that project radially outwardly from the yarn create mini short-circuits between the yarns, as can be seen in FIGS. 3A and 3B. Lower background noise in the data permits much more straightforward signal processing.

In addition, different properties of specific types of yarn can be used to tailor the extensibility of the yarn. For example, in some embodiments described later, a 2-ply yarn having a particular overall yarn count is seen to provide a better repeatability than a 1-ply yarn of the same overall yarn count. In other words, extensibility of yarns is improved by reducing the homogeneity of the yarn, as this may reduce friction and improves the slipping of yarns in contact with one another, so that the stretch of the fabric is reduced at the same time as reducing extensibility.

The above embodiments relate to the knitted textile sensor itself. The boundary zone that extends between the textile sensor and the support fabric may also be designed to improve the repeatability of the textile. As noted above, the boundary zone comprises overlapping stitches from the textile sensor and support fabric so that stitches in the wales in the boundary zone alternate between stitches formed by the yarn from of the textile sensor and stitches formed by the yarn from the support fabric, and in the boundary zone of FIG. 5, the stitch pattern is 100% single jersey stitches. In an embodiment of the invention to reduce deformation of the sensor during use and, thus, to improve repeatability, an increased percentage of tuck stitches may be included within the boundary zone. In these embodiments, the boundary zone comprises a stitch pattern having a combination of tuck and jersey stitches to connect the textile sensor and the support fabric. The arrangement of the tuck stitches may be staggered, as is explained below in relation to FIG. 6.

The incorporation of tuck stitches in the boundary zone further reduces unwanted stretching in the wale direction. This is important in the boundary zone as the effective working range of the textile sensor can be defined and controlled by the mechanical properties of the boundary zone. In this way, the boundary zone acts as a reinforcing structure within the textile and maintains the position and aspect ratio of the textile sensor, thereby preventing it from elongating beyond its useful working range.

In addition, the embodiments described herein are resistant to deformation caused by repeated laundering either by hand or machine washing. In particular embodiments of the invention the knitted textile sensors are able to maintain a working sensor range over at least ten washing cycles, suitably at least 50 washing cycles and up to at least 100 washing cycles. A typical washing cycle will include a washing step, a rinsing step and a drying step.

An example of how the boundary zone 16 may incorporate tuck stitches 120 is shown in FIG. 6. In this embodiment, the courses of the support fabric 14 on either side of the textile sensor 12 and the courses of the textile sensor 12 have been separated out, so that the course overlaps in the boundary zone 16 are more clearly visible. The regions of overlap indicating the boundary zone 16 are highlighted in this figure with a dotted white box. In FIG. 6, the textile sensor (located in the central region of the textile) 12 has a stitch pattern with 50% jersey stitches and 50% tuck and miss stitches. The support fabric 14 is comprised of a 100% jersey stitch textile. In the boundary zone 16, as described above, the overlap between wales has a stitch pattern comprising exclusively jersey stitches 100 and tuck stitches 120. The tuck stitches 120 are staggered within the boundary zone so a tuck stitch formed by the yarn of a first course of the support fabric around the yarn of a first course of the textile sensor is followed by a tuck stitch formed by the yarn of the first course of the textile sensor with the yarn of the second course of the support fabric. This is visible in both the left and right boundary zones 16 of FIG. 6.

In incorporating more tuck stitches into the boundary zone and the sensor than in the support fabric, the region of the textile covering the boundary and sensor is stiffer, i.e. is less able to stretch, than the support fabric. The improved stiffness is important in ensuring that the boundary and sensor are able to return to their original positions during relaxation. A lower stiffness in the support fabric is desirable as it ensures that the rest of the textile can stretch to accommodate the application for which it is being used.

Although not shown in the figures of this application, in embodiments where a boundary zone is further provided that extends in the course direction along the upper and lower edges of the textile sensor, the connection may be made using single jersey stitches for stability of the textile sensor.

The support fabric may also be configured to improve the repeatability, either as a separate change or in combination with one or more of the other features listed above. In specific embodiments, the support fabric may be comprised within, or constitute, a garment or bandage. In such instances, the support structure must provide a dual role of performing as the intended garment or bandage as well as providing mechanical and positional support to the integrated sensor. The structure of the support fabric is desired to reduce creasing in the textile sensor to improve the repeatability of its measurements. The support fabric is also designed to return the aspect ratio of the textile sensor to or close to an original aspect ratio after elongation. Accordingly, in particular embodiments, several features may be provided to reduce creasing or waisting and to maintain the aspect ratio of the sensor.

A first of these features is the inclusion of particular stitch structures within the stitch pattern of the support fabric. In particular, the incorporation of a plurality of miss stitches can create a ribbed effect in the fabric that reduces creasing. Hence, in an embodiment of the invention a combined stitch pattern is provided having a majority of single jersey stitches (e.g. at least 50%) and a minority of miss stitches (e.g. less than 50%).

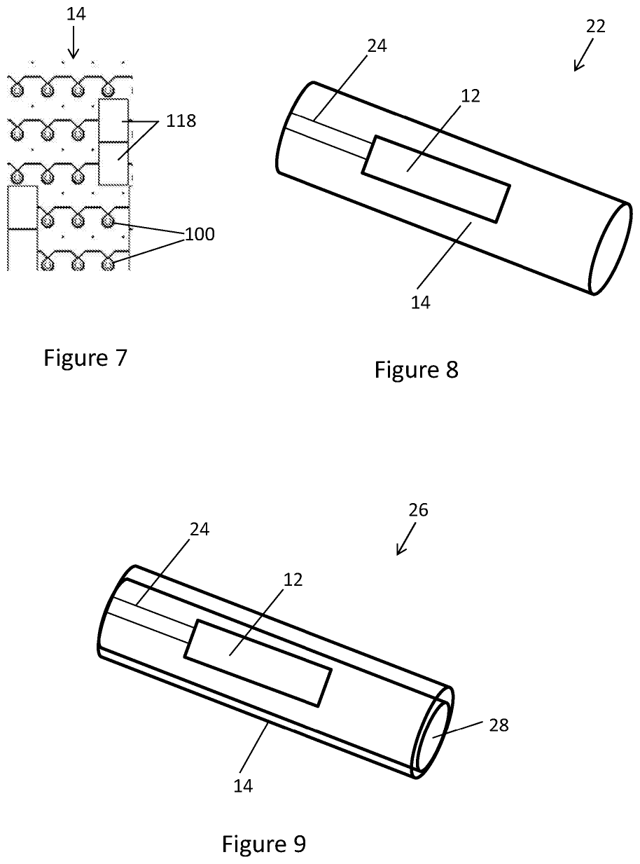

In particular embodiments, the stitch structures may comprise double miss stitches. An example of a portion of the support fabric incorporating double miss stitch structures within an otherwise jersey stitch pattern is shown in FIG. 7. The miss stitches of the double miss stitch structure are provided in the same wale in consecutive courses—i.e. they are ‘stacked’ on top of one another. This structure reduces creasing in the textile in the course direction, and introduces tension in the course direction to maintain the coursewise width of the textile sensor during elongation in the wale direction.

In some embodiments, the textile is formed into a tubular garment (e.g. a sock, sleeve, cuff or support bandage) 20, with the support fabric 14 forming a substantial part of the tubular garment 20. This also reduces waisting by maintaining tension across the surface of the textile sensor 12. An example of a tubular garment is shown in FIG. 8, where the textile sensor 12 and connection means in the form of conductive tracks 24 are provided and extend longitudinally within the substantially tubular support fabric 14.

Another improvement is found in the provision of a stabilising layer. The stabilising layer is provided on one side of the support fabric and sensor, and is attached to the support fabric and/or sensor. The stabilising layer may be a knitted textile or other fabric. Its presence stabilises the movement of the textile in the direction of elongation and prevents bunching of the support fabric and textile sensor. An example of a stabilising layer 28 is shown in FIG. 9, provided within a tubular garment 26. Additionally, when the textile sensor 12 is intended for application adjacent or proximate to a skin surface of a subject or other conductive surface, the stabilising layer 28 insulates the textile sensor 12 from the conductive surface, thereby reducing noise in the data obtained from the textile sensor 12. The stabilising layer 28 may incorporate a more open stitch pattern or fabric having more space between fibres to enable better heat dissipation through the garment 26.

In general, the support structure, whether including a support fabric and stabilising layer or just a support fabric, is preferably relatively elastic when compared to the sensor, in order to allow the structure to maintain the position of the textile relative to a user, and to permit movement of, for example, the limb to which it is applied. In embodiments where the garment is used to measure movement of the elbow joint (e.g. flexion and extension), and where elongation and resistance is correlated with movement from a flat arm to a bent arm, the sensor's stiffness permits it to stretch around and maintain its position relative to the elbow. Consequently, the support fabric is sufficiently elastic to permit relative movement of the rest of the joint. If the support fabric were stiffer than the sensor, movement of the elbow joint would be restricted, and the sensor would not maintain its position relative to the joint. The positioning of the sensor is dependent upon the supporting structure to ensure that optimal positioning within the plane of movement of the joint is maintained. The stabilising layer may be made from a fibre having highly elastic synthetic fibres. Examples may be Lycra®, which is also known as Spandex or elastane. The Lycra® may be combined with nylon or polyester to improve the characteristics of the fabric for use as a stabilising layer. Lycra® or nylon-lycra or polyester-lycra yarn may also be used to form the support fabric.

Each of the above-listed features, whether used in a textile alone or in combination, improve the repeatability of the elongation of the textile sensors to enable useful measurement of movement of an item to which the textile sensor is attached. A highly repeatable system is achieved by combination of each of the above features. This system comprises a textile sensor having a stitch pattern having 50% single jersey stitches and 50% tuck and miss stitches of which the majority are tuck stitches. The textile sensor is connected to a support fabric at a boundary zone, the boundary zone comprising single jersey and tuck stitches. The support fabric comprises single jersey stitches with miss stitch structures, preferably double miss stitch structures, therein. The textile as a whole is arranged into a tubular form, to be extended along a longitudinal axis of the tube, and incorporates a stabilising layer therein that is attached to the support fabric.

The smart sleeves discussed above show particular utility in rehabilitative and sports purposes with portable power and acquisition units. The smart sleeves show particular advantages as they retain their properties after multiple cycles of washing, allowing them to be used domestically as well as in clinical situations.

The following examples were performed to indicate the usability of textile sensors and textiles having some or all of the above features.

### EXAMPLES

### Example 1

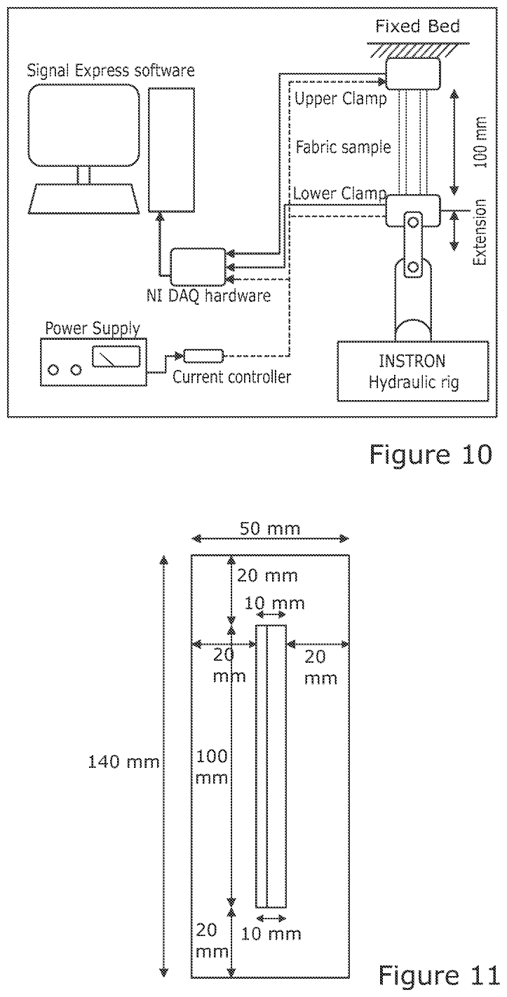

To investigate the hysteresis of textiles and textile sensors having some of the features described above, the experimental equipment of FIG. 10 was used with samples of textiles in the form shown in FIG. 11. The experimental equipment comprised a data acquisition system, a power supply and current controller, a pair of clamps between which textile samples are placed and one of which is fixed, and a hydraulic rig for elongating the sample by moving the clamp that is not fixed.

The textile samples, as exemplified in FIG. 11, comprised a textile sensor centrally arranged within a support fabric. The whole sample was 140 mm long while the textile sensor is 100 mm long. The clamps covered the 20 mm of support fabric above and below the textile sensor. The samples were 50 mm wide, with the textile sensor portion being 10 mm wide.

The samples used differed in their stitch patterns in the textile sensor and the electrically conductive yarn used in the sensor. The support fabric was constructed using nylon yarn.

Each sample was mounted into the clamps. The hydraulic rig elongated and relaxed the samples 12 mm in the wale direction through 1500 cycles at a rate of 0.9 Hz. The response of the textile sensor through its change in contact resistance was recorded using the data acquisition system.

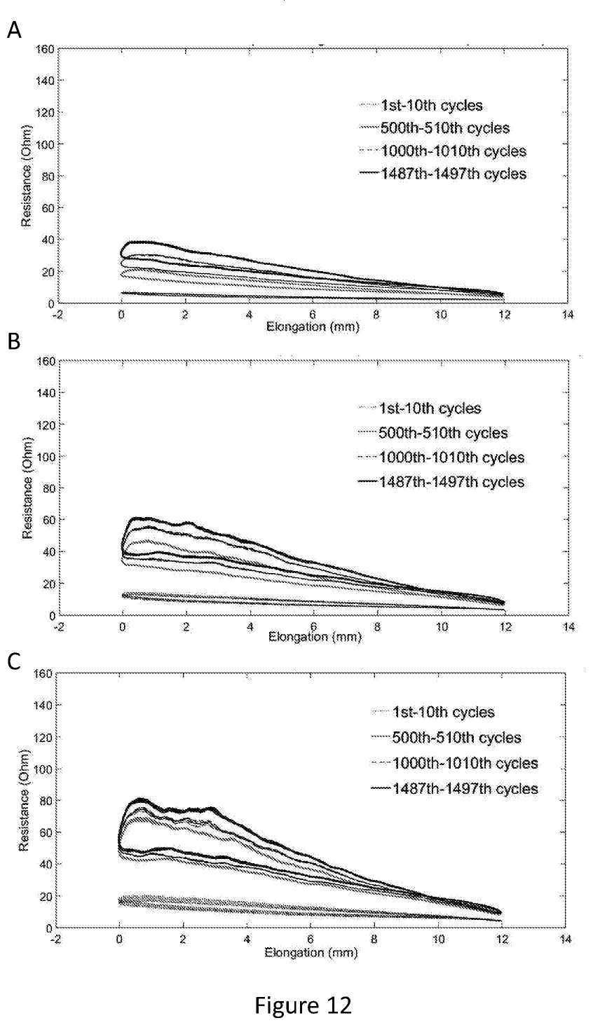

The results for a first textile sample are shown in FIGS. 12A to 12C. FIG. 12A represents an initial test of the fabric. After the trial of FIG. 12A, the textile was removed from the clamps, rested, and then re-tested. The results of these repetitions of the test are shown in FIGS. 12B and 12C.

The textile sensor of the first textile sample comprised a first stitch pattern having 50% single jersey stitches, and 50% miss and tuck stitches with a majority of this 50% being tuck stitches. The textile sensor of the first sample included a first yarn comprising single ply multifilament yarn.

In FIG. 12A, results for sets of cycles are shown. The relationship between resistance and elongation during the elongation and relaxation of the first textile sample are shown for the 1^{st} to 10^{th}, 500^{th} to 510^{th}, 1000^{th} to 1010^{th}, and 1487^{th} to 1497^{th} cycles. As can be seen, during each set of cycles, the hysteresis curve exhibited by the sample is highly repeatable. Over time, the resistances increase as the textile changes shape, as can be seen in the change in shape of the hysteresis pattern between the 1^{st} to 10^{th} cycles and the later sets of cycles. However, within small sets of cycles, the relationship between resistance and elongation length in both the elongation and the relaxation of the textile is highly similar, and so can be utilised in measuring elongation.

In the textile sample, when completely relaxed, i.e. at 0 mm elongation, the contact resistance is at a first level. The contact resistance changes during elongation to a second, lower level at maximum elongation, i.e. 12 mm.

Not only is the action of the textile highly repeatable during a first test, but as can be seen from FIGS. 12B and 12C, carried out on the same sample as FIG. 12A after resting the textile sample, the textile sample maintained its repeatability, especially in the early cycles. In later cycles, the resistance in the textile sensor was higher in each test than in previous tests but the shape of the hysteresis curve remained broadly consistent.

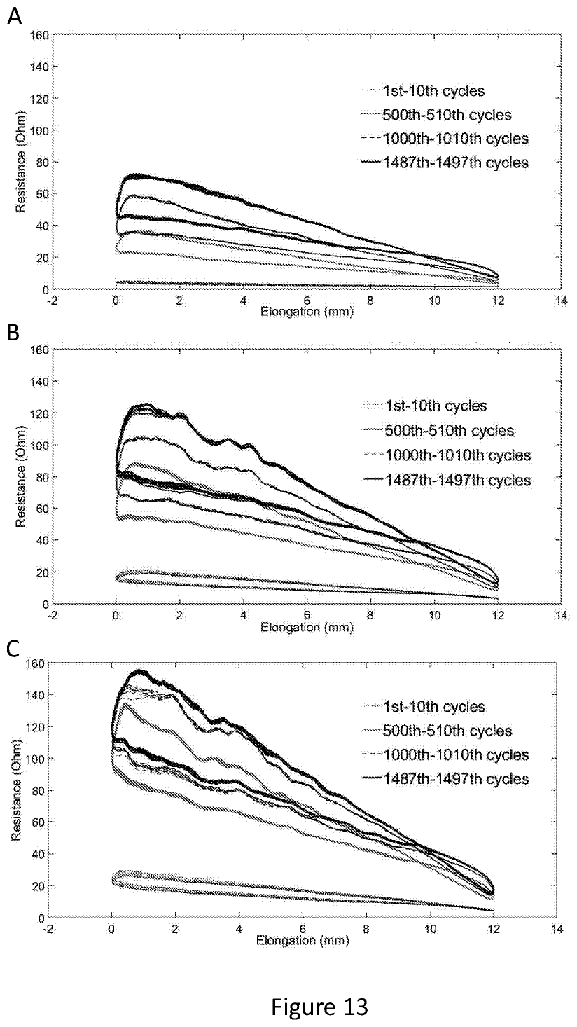

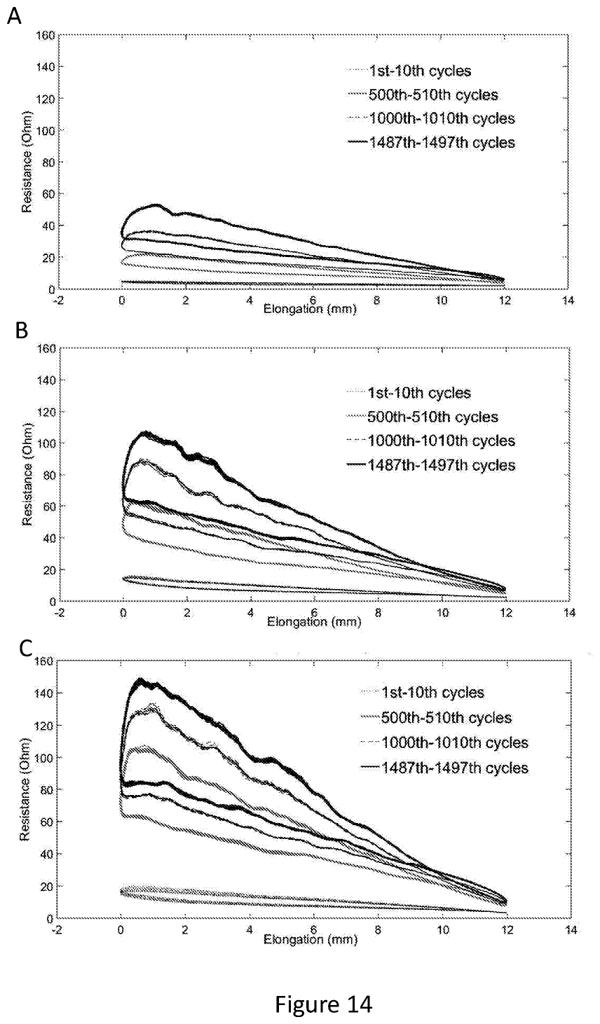

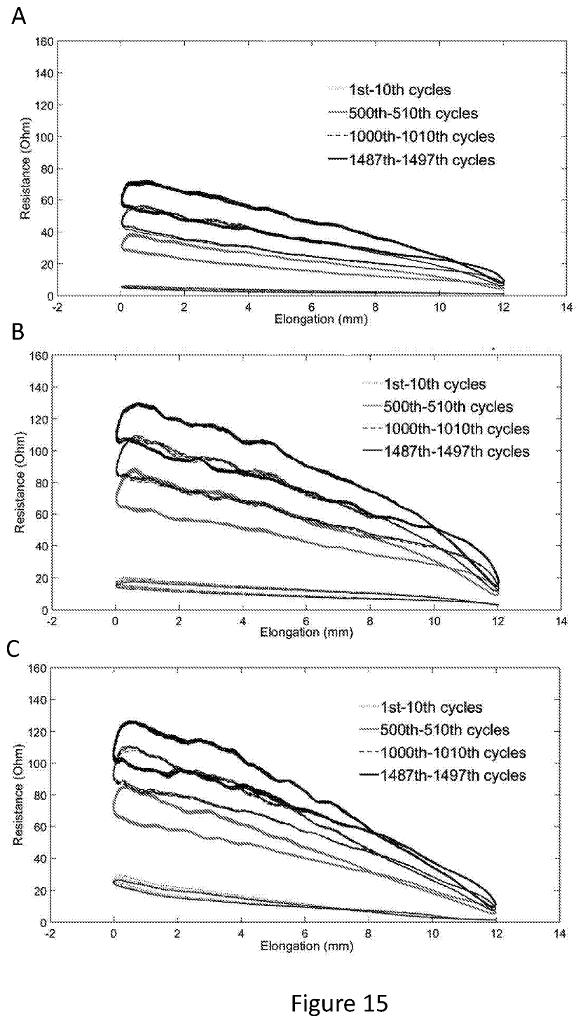

The results for a second, third, and fourth textile sample are shown in FIGS. 13A to 13C, 14A to 14C, and 15A to 15C. As in FIGS. 12A to 12C, FIGS. 13A, 14A, and 15A represent an initial trial of their respective samples, while FIGS. 13B, 13C, 14B, 14C, 15B, 15C represent repetitions of the trial using the same sample that has been rested for a while.

The textile sensor of the second textile sample comprised the first stitch pattern, i.e. the same stitch pattern as the first textile sample. The textile sensor of the second sample included a different second yarn comprising a double ply multifilament yarn having the same yarn count as the first yarn.

The textile sensor of the third textile sample comprised the first yarn and a different, second stitch pattern. The second stitch pattern comprised 50% single jersey stitches and 50% miss and tuck stitches with a majority of this 50% being tuck stitches but the percentage of tuck stitches was lower in the second stitch pattern than in the first stitch pattern. The stitch patterns comprise 50% single jersey stitches and 50% miss and tuck stitches with a majority of tuck stitches. Suitably, the stitch pattern comprises at least 5% miss stitches and 45% tuck stitches, and more suitably, comprises miss stitches between 15% and 24%, with the remaining percentage of stitches, i.e. between 35% and 26%, being tuck stitches.

The textile sensor of the fourth sample comprised the second yarn and the second stitch pattern.

In other words, the second sample differed from the first sample by its yarn, the third sample differed from the first sample by its stitch pattern, and the fourth sample differed from the first sample by its yarn and its stitch pattern. All the textile samples had the same stitch pattern within the support fabric.

As can be seen in FIGS. 13A to 15C, these second, third, and fourth textile samples also exhibit a highly repeatable hysteresis over small sample sets, although it is seen that the first sample has the smoothest curves with the lowest resistances.

Accordingly, textiles using stitch patterns having 50% jersey stitches split with 50% majority tuck and minority miss stitches and matched with yarns with a low extensibility are shown to be particularly useful where a repeatable textile hysteresis pattern is desirable.

There are a number of uses for such a textile. The textile may be formed into a tubular garment for wearing by a patient performing physical rehabilitation exercises on a joint. The textile sensor may be arranged over the joint and configured to be at 0 mm of elongation when the joint is bent or to be at 0 mm of elongation when the joint is straight, depending on whether the sensor is positioned on the inside or the outside of the joint. By way of example the ‘inside’ for the elbow represents the ventral surface and the ‘outside’ represents the dorsal surface. Whereas, for the knee joint the opposite is true.

When the joint completes a cycle of flexure such that it is extended and returned, the textile sensor will produce output signals similar to those shown in FIGS. 12 to 15. Using a first elongation as a calibration elongation, the system may subsequently track full extensions of the joint to ensure that the full extension of the joint is achieved by the patient correctly.

Elbow joints, knee joints, wrist joints, knuckles, ankles, and any other extendible joint that flexes predominantly within a single plain of movement may be monitored using a garment incorporating the textile described herein.

Textiles described herein may be returned to a base state or ‘reconditioned’ by washing and drying the textile.

### Example 2

Textile-based strain sensors combine wearability with strain sensing functionality by using only the tensile and electrical properties of the threads they are made of. In this study, two conductive sleeves were manufactured for the elbow and three for the knee using a Santoni circular machine with different combinations of elastomeric and non-elastomeric yarns. Linearity, repeatability and sensitivity of the sleeves resistance with strain were compared during 5 repetitive trials, each of them consisting of 4 sequences of 50 joint flexion-extension cycles. All knitted conductive sleeves registered motion over 1000 cycles, proving their suitability for joint motion tracking. In addition, sleeves whose inner layer was made only with nylon, exhibited the highest sensitivity and predictability of changes (i.e. a linear trend of the non-elastic deformation). On the other hand, sleeves whose inner layer was made with lycra and polyester or lycra and nylon showed a more balanced performance in terms of linearity, sensitivity and repeatability either for low or high number of cycles. Based on requirements, the presented sleeves can be used for rehabilitation both in healthcare and sports.

Human motion detection is important for treating medical conditions (e.g. musculoskeletal diseases), for rehabilitating after injuries (e.g. stroke), for improving athletic performance and for assessing the design of orthosis and prosthesis. Motion analysis based on conventional strain sensors relies on devices made of rigid materials (e.g. metals or semiconductors), which are typically bulky, hard-to-wear and withstand small strain (less than 5%). An unobtrusive solution, comfortable to wear and feasible to fit the human body, is offered by textile-based strain sensors. They combine wearability and high flexibility with large strain sensing functionality [1] and provide strain measurements based on the electrical properties of the threads they are made of. Therefore, wearable textile strain sensors are increasingly important to track movements of the human body, either small strain (e.g. respiration [2]) or large strain (e.g. 55% deformation during walking [3]), to assist in sports and remote health monitoring [4], and for soft robotics [5].

Fabric strain sensors are proposed as wearable devices for measuring joint angles. To monitor the desired knee flexion angle on a wearer, [6] integrated a polypyrrole-coated nylon-lycra sensor into a base sleeve using press-studs. [7] attached an elastic conductive webbing made of polyamide fibres coated with carbon particles and polyester yarn to a woven fabric, which was used for monitoring the flexion angle of elbow and knee. However, both wearable solutions did not embed the sensor into the garment but attached it on its surface as an external sensing element.

Conductive yarns alone offer moderate levels of strain. Therefore, to facilitate sound tensile recovery, the grade of elasticity in fabrics can be increased by knitting conductive yarns along with elastomeric yarns. Depending on the type of elastic yarn used (e.g. single or double-covered, core—spun), the tensile and conductive properties of knitted strain sensors can be affected and thus their sensitivity performance [8].

The sensing mechanism in textile strain sensors upon stretching/recovery is due to different factors correlated to each other: (1) length variation of the conductive yarn contributing to the length-related resistance (according to Ohm's law [9]), (2) structural deformations of the loop geometry affecting the number of contact points and contact pressure and, thus, the contact resistance (according to the Holm's contact theory [10]), and (3) change in the conduction path due to transformation of the equivalent electrical network associated to the fabrics structure [11].

In this example, unlike the flat-bed knitting technology employed in previous works for producing strain sensing fabric [12], a circular knitting machine was used for manufacturing and integrating the textile conductive sensor into the garment during the same knitting process. The outcome was knitted conductive sleeves, which were sensitive to the strain caused by the motion of the wearer's joints. Different combinations of elastomeric and non-elastomeric yarns were chosen and their overall sensing properties compared during repetitive joint motion. For their properties, the knitted conductive sleeves will be referred to as smart sleeves in the rest of the paper.

The objectives of this work are the characterisation of the electrical properties of knitted conductive sleeves during repetitive flexion-extension cycles and their performance evaluation when different materials are used.

### Experimental Set-Up

### A. Materials

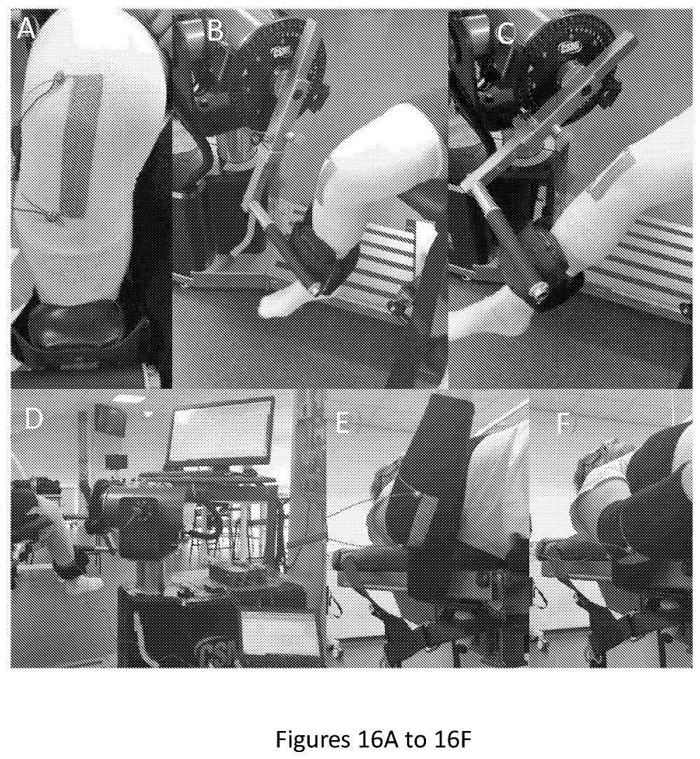

Two elbow sleeves and three knee sleeves (Footfalls and Heartbeats (UK) Limited) in FIGS. 16(a) to 16(f) were manufactured on a Santoni X-machine, a single cylinder intarsia machine with 4 feeds, 12 gauge, and 144 needle count. Each sleeve comprised a knitted conductive sensor, which was 90 mm×15 mm (height×width) and made of silver plated nylon. The response of the sensing area to the joint motion (i.e. flexion-extension) was changes in the fabric conductivity due to the stretching-recovery of the fibres. The novelty from previous research on knitted strain sensors during cyclic loading [13] was replacing stainless steel spun staple fibre yarn (i.e. short fibres) with silver multifilament yarn (i.e. 100% continuous conductive fibres), which significantly improved the sensor response in terms of repeatability over time [14]. The stitch pattern of the sensor used in this example is described in [15]. To avoid any direct contact between the skin and the conductive area of the sensor, each sleeve included an inner layer which was sewn in the interior part of the garment. The materials used for the sensor, the sleeve and the inner layer are described in Table I for the elbow (E prefix) and knee (K prefix) sleeves.

TABLE I
Smart sleeves and their composition

| Smart | Materials | | |
|---|---|---|---|
| Sleeves id | Sensor | Sleeve | Inner layer |
| E4 | Silver plated | Lycra (22 dtex) | Nylon (600 dtex) |
| | nylon (300 dtex) | | |
| E5 | Silver plated | Lycra (22 dtex) | Lycra (20 dtex), |
| | nylon (300 dtex) | | Polyester (300 dtex) |
| K1 | Silver plated | Lycra (20 dtex) and | Nylon (600 dtex) |
| | nylon (300 dtex) | Nylon (600 dtex) | |
| K3 | Silver plated | Lycra (20 dtex) | Lycra (20 dtex), |
| | nylon (300 dtex) | | Nylon (600 dtex) |
| K4 | Silver plated | Lycra (20 dtex) | Lycra (20 dtex), |
| | nylon (300 dtex) | | polyester (197 dtex) |

### B. The Equipment

The equipment used for investigating the sensing properties of the smart sleeves during flexion-extension trials consists of the HUMAC NORM machine (HUMAC2015®/NORM™) and a dedicated acquisition system. In this study, the dynamometer was employed for offering repetitive knee/elbow joint movement according to a set angle/speed. The equipment in FIG. 16(d) includes the HUMAC NORM Isokinetic Machine, a power supply, an electronic board working as a constant current generator, a data logger (NI USB 6003) and a PC with the MATLAB® Analog Input Recorder. The constant current generator was set to 10 mA and data acquisition was 1 kHz.

### Method

### A. Test Protocol

Written consent was obtained by a healthy volunteer after the test protocol was approved by the University of Nottingham Faculty Research Ethics Committee. The dynamometer was set in continuous passive motion (CPM) operating mode, i.e. regardless of whether the user was performing a concentric or an eccentric contraction (i.e. muscle shortening/lengthening). This choice stemmed from a research purpose and not a rehabilitative goal.

The dynamometer constantly moved the joints through a controlled range of motion (0°-90°), with 0° being calibrated for the full extension. The constant rotation rate was +60°/s during the joint flexion (from 0° to 90°) and −60°/s during the joint extension (from 90° to 0°). These values were chosen considering the joint operating range and speed of a real application. For each smart sleeve in Table I, the testing procedure consisted of 5 trials, each of them comprising 4 sequences of 50 flexion-extension cycles with 10 second rest in between. Each trial was repeated after a 5 minute rest interval without modifying the sensor and the parameters in the HUMAC software.

### B. Signal Processing

Fabric electrical voltage and current, joint angle and rotation speed were simultaneously collected with the acquisition system of FIG. 16(d) and post-processed in MATLAB. The calculated resistance was filtered with a Savitzky-Golay filter, whose frame size (f=317) was adapted to the stretch-recovery speed of the fabric sensor and the polynomial order (N=1) to the observed shape of the signal. Next, peak analysis on the filtered resistance was conducted, with the maximum and minimum resistance values being detected for each cycle and associated with the motion performed. Subsequently, interpolation curves passing through the maximum and minimum filtered values were established, to compare at every instant maximum and minimum points which were originally separated from each other by half a period. This allowed to calculate the peak-to-peak span (by subtracting the interpolated minimum curve from the interpolated maximum one), which provided information in terms of sensitivity of the fabric-transducer.

### C. Linearity Evaluation

One of the phenomenon influencing the performance of fabric sensors is the fibre-fibre slippage. Fabrics are fibrous materials which, under strain, undergo often irreversible slippage between fibres, resulting in changes in dimensions of the assembly and, therefore, overall non-linear deformation of the fabric. As a result, non-linearity and hysteresis are expected in the sensor response to the performed motion.

A study of hysteresis was conducted to quantify the sensor non-linearity during repetitive range of motion. In particular, the mean hysteresis areas, Hₘ, and their relative change were calculated for all candidates of Table I at 200 and 1000 cycles (for rehabilitative and sports applications, respectively):
Mean hysteresis area of the initial 50 cycles H_{m_50})

$${H}_{{m}_{50}} = \frac{{\sum }_{i = 1}^{50} {H}_{i}}{50} , \qquad ( 1 )$$

Initial 200 cycles relative change (h_{_200i}):

$${h}_{20 {0}_{i}} = ( {\sum }_{i = 150}^{200} \frac{{H}_{i}}{50} - {H}_{{m}_{50}} ) / {H}_{{m}_{50}} , \qquad ( 2 )$$

Final 200 cycles relative change (h_{_200f}):

$${h}_{20 {0}_{f}} = \frac{{\sum }_{i = 950}^{1000} {H}_{i} / 50 - {\sum }_{i = 800}^{850} {H}_{i} / 50}{{\sum }_{i = 800}^{850} {H}_{i} / 50} , \qquad ( 3 )$$

Overall relative change over 1000 cycles (h_{_1000}):

$${h}_{1000} = ( {\sum }_{i = 950}^{1000} \frac{{H}_{i}}{50} - {H}_{{m}_{50}} ) / {H}_{{m}_{50}} . \qquad ( 4 )$$

In addition, to measure the predictability of the non-linear deformation, a curve fitting with a linear profile was performed in each sequence of every trial and the corresponding R² coefficients were extracted. In this paper, R² over 1000 cycles was reported for the initial and last 50 cycles, R²_{H_50i} and R²_{H_50f}, respectively.

### D. Repeatability Evaluation

The fabric deformation over time affects also the repeatability of the electrical response of textile sensors during repetitive elongation-recovery cycles. To measure the repeatability of the response of smart sleeves during flexion-extension trials, the standard deviation of the hysteresis areas, O_{H}, and its relative change was calculated:
During the initial 50 cycles (R_{_50i}):

R₅₀_{ᵢ}=σ_{H}(H₁,H₂. . . H₅₀), (5)
During the last 50 cycles (R_{_50f}):

R₅₀_{_{f}}=σ_{H}(H₉₅₀,H₉₅₁. . . H₁₀₀₀), (6)
Overall relative change over 1000 cycles (r_{_1000}):

r₁₀₀₀=(R₅₀_{_{f}}−R₅₀_{ᵢ})/R₅₀_{ᵢ}, (7)

### E. Sensitivity Evaluation

The sensitivity of the smart sleeves was determined as the ratio between the peak-to-peak span of the fabric electrical resistance and the span of the joint angle. As the peak-to-peak span increases with the non-elastic fabric deformation and thus with cycles, so does sensitivity. Therefore, it was also important to characterise the linearity of this increasing trend. To compare performance, the following sensitivity values were calculated:
Sensitivity of the first (S₁) and last cycles (S₁₀₀₀),
Mean sensitivity of initial 200 cycles (S_{m_200i}):

$${S}_{{m}_{20 {0}_{i}}} = \frac{{\int }_{1}^{200} S ( c ) d c}{200} , with c number of cycles \qquad ( 8 )$$

Mean sensitivity of final 200 cycles (S_{m_200f}):

$${S}_{{m}_{20 {0}_{f}}} = \frac{{\int }_{800}^{1000} S ( c ) d c}{200} , with c number of cycles \qquad ( 9 )$$

Overall relative change over 1000 cycles (s_{_1000}):

S₁₀₀₀=(S₁₀₀₀−S₁)/S₁, (10)
Linear sensitivity trend for initial 200 cycles (R² s_{_200i}),
Linear sensitivity trend for final 200 cycles (R²s_{_200f}).

### F. Sleeves Performance Comparison

To allow a straightforward performance comparison, radar plots were drawn based on evaluation criteria (high values) for linearity (1/H_{m_50}, R²H_{_50i}, R²_{H_50f}, 1/h_{_200i}, 1/h_{_200f}, 1/h_{_1000}), sensitivity (S₁, S_{m_200i}, S_{m_200f}, 1/_{s_1000}, R²_{S_200i}, R²_{S_200f}) and repeatability (1/R_{_50i}, 1/R_{_50f}, 1/r_{_1000}).

### Result and Discussion

### A. Overall Electrical Characterisation

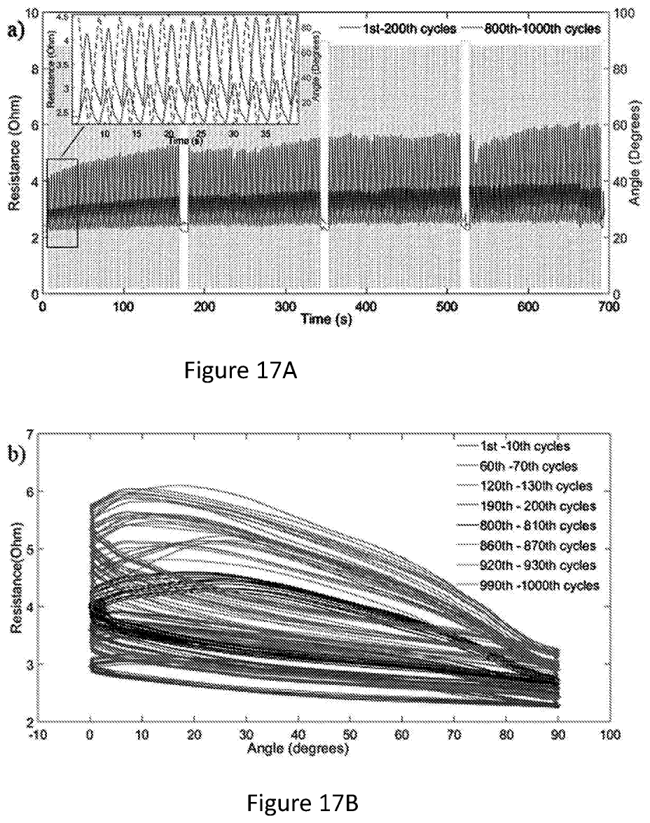

The electrical response of all smart sleeves during repetitive joint motions was studied. FIG. 17(a) shows the resistance variation with the angle performed for the E5 elbow sleeve during trial 1 (1st to 200th cycle) and trial 5 (800th to 1000th cycle). The textile sensor detected the type of motion executed, i.e. flexion (from maximum to minimum resistance values) and extension (from minimum to maximum resistance values) and exhibited reliable performance up to 1000 cycles. These results highlighted the potential of smart sleeves (and of the knitted sensor) as wearable textile strain sensors and their suitability for joint motion tracking. FIG. 17(b) shows the hysteresis during equivalent cycles.

### B. Effect of Materials on Sleeves Performance

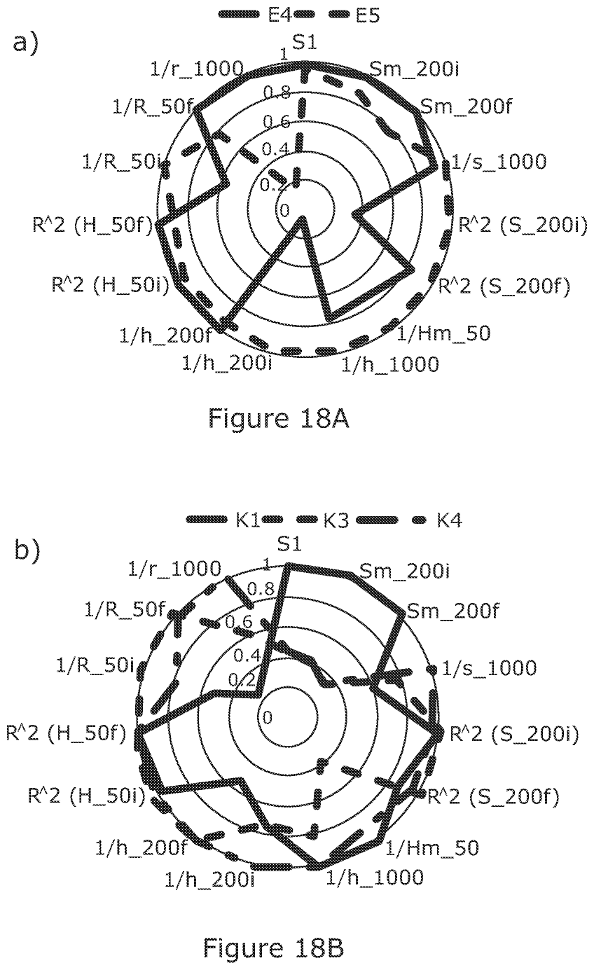

Materials used for sleeve and inner layer have an effect on the overall performance. Elbow sleeves whose inner layer was made only with nylon (i.e. E4, FIG. 18(a)) exhibited higher overall sensitivity (S₁, S_{m_200i}, S_{m_200f}), higher linearity (1/h_{_200f}) and repeatability (1/R_{_50f}, 1/r_{_1000}) after a considerable number of cycles and a more predictable (i.e. linear) deformation trend (R²H_{_50i} and R²H_{_50f}). This is due to higher stiffness of nylon compared to lycra/polyester which delays the irreversible fibres deformation responsible for loss of linearity. On the other hand, elbow sleeves whose inner layer was made with lycra and polyester (i.e. E5) show better overall performance in terms of linearity, sensitivity and repeatability both for low (1/H_{_m50}, 1/h_{_200i}, R²_{s_200i} 1/R_{_50i}) and high number of cycles (1/h_{_1000}, R²S_200f, 1/S_{_1000}).

Knee sleeves, whose inner layer consisted of nylon only (i.e. K1, FIG. 18(b)), exhibited a higher sensitivity (S₁, S_{m_200i}, S_{m_200f}, R²s_200f). Knee sleeves with lycra and nylon (i.e. K3) or lycra and polyester (i.e. K4) in the inner layers presented similar good performance in terms of linearity (1/h_{_1000}, 1/h_{_200f}, R²H_{_50i}, R²H_50f), sensitivity and repeatability, with K4 having a more comfortable wearability due to polyester.

### CONCLUSION

This example studied performance of knitted conductive sleeves (with the textile conductive sensor integrated into the garment) and proved their suitability as textile strain sensors for joint motion tracking. An evaluation method was proposed (based on specific parameters per application) and radar plots were used for straightforward comparison. Knit candidates can be selected based on durability of the overall sensing properties, low hysteresis and comfort (i.e. E5, K3 and K4) or high sensitivity response (i.e. E4 and K1). This indicates that the smart sleeves can be used for rehabilitative and sports purposes with portable power and acquisition units.

### Example 3

The degradation of a knitted conductive sensor as described above was assessed having undergone up to 100 washes in a domestic washing machine. The goal was to evaluate whether the knitted sensor remains functional following standard washing and drying procedures (in conformance with the British standard washing procedure protocol, [16]). This will provide an estimation of the lifespan of the sensor during normal use. In particular, washability and long-term sensing properties are important requirements to be maintained by the knitted sensor integrated into a garment and used in wearable applications (e.g. human motion monitoring).

Washability and long-term reliability of the knitted structure presented herein were analysed once embedded into an elbow sleeve in correspondence of the joint. The sensorised sleeve was manufactured with the Santoni-X knitting machine, was made of Lycra (22 dtex) and included an inner layer made of Lycra (20 dtex) and polyester (300 dtex). Tests were conducted on a user wearing the sleeve and performing 50 flexion-extension cycles (within a comfortable range of motion and not at a set speed) after an increasing number of washes.

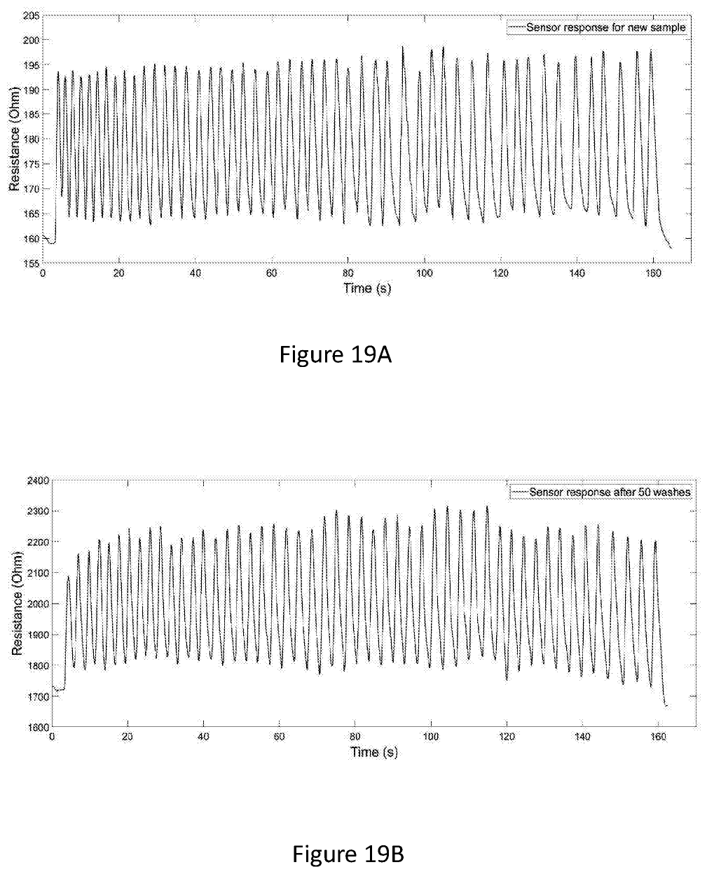

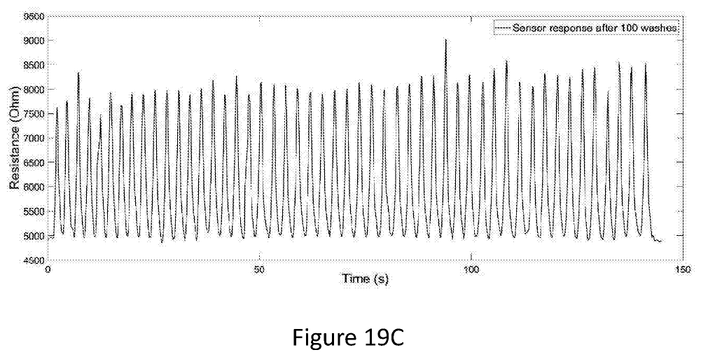

FIGS. 19A to 19C show the electrical response of the sensorised sleeve during 50 flexion-extension cycles for a new sample (FIG. 19A), a 50-time washed sample (FIG. 19B) and a 100-time washed sample (FIG. 19C). The increasing resistance seen after an increased number of wash cycles is mainly due to the fibres' relaxation as a result of the washing detergent use [17].

It can be noticed that after 50 and 100 washes, the electrical patterns replicating flexions (from maximum to minimum resistance values) and extensions (from minimum to maximum resistance values) are still clear, distinguishable and repeatable.

This highlights the sensor's long-term ability of detecting, distinguishing and counting the type of motion performed after 100 washes and, therefore, its suitability for commercial applications.

### REFERENCES

[1] S. Seyedin et al., “Textile strain sensors: a review of the fabrication technologies, performance evaluation and applications,” Mater. Horiz, vol. 6, pp. 219-249, 2019.
[2] R. Paradiso et al., “WEALTHY, a Wearable Health-Care System: New Frontier on E-textile,” Journal of Telecommunications and Information Technology, vol. 4, pp. 105-113, 2005.
[³] T. Yamada et al., “A stretchable carbon nanotube strain sensor for human-motion detection,” Nature Nanotechnology, vol. 6, pp. 296-301, 2011.
[4] S. Majumder et al., “Wearable Sensors for Remote Health Monitoring,” Sensors vol. 17, no. 1, p. 130, 2017.
[5] Y. Cheng et al., “A stretchable and highly sensitive graphene-based fiber for sensing tensile strain, bending and torsion,” Advanced Materials, vol. 27, no. 45, pp. 7365-71, 2015.
[6] B. J. Munro et al., “The intelligent knee sleeve: A wearable biofeedback device,” Sensors and Actuators B: Chemical, vol. 131, no. 2, pp. 541-547, 2008.
[7] T. W. Shyr et al., “A Textile-Based Wearable Sensing Device Designed for Monitoring the Flexion Angle of Elbow and Knee Movements,” Sensors, vol. 14, no. 3, pp. 4050-4059, 2014.
[8] K. R. Rafiu et al., “Influence of Rib Structure and Elastic Yarn Type Variations on Textile Piezoresistive Strain Sensor Characteristics,” Fibres and Textiles in Eastern Europe, vol. 26, no. 5, ed. 131, pp. 24-31, 2018.
[9] J. C. Shedd and M. D. Hershey, The history of Ohm's Law, The Popular Science Monthly Bonnier Corporation, pp. 599-614, 1913.
[10] R. Holm, Electric Contacts—Theory and Application. 4th edition, Springer-Verlag, 1967.
[11] C. lsaia, “Characterisation of knitted conductive textiles for wearable motion sensor applications,” PhD thesis, University of Nottingham, U K, 2018.
[12] O. Atalay et al., “Textile-Based Weft Knitted Strain Sensors: Effect of Fabric Parameters on Sensor Properties,” Sensors, vol. 13, no. 8, pp. 11114-11127, 2013.
[13] C. lsaia, D. S. McNally, S. A. McMaster, and D. T. Branson, “Effect of mechanical preconditioning on the electrical properties of knitted conductive textiles during cyclic loading,” Textile Research Journal, vol. 89, no. 3, pp. 445-460, 2019.
[14] C. lsaia, S. A. McMaster, GB Patent Application (In development), January 2020.
[15] S. A. McMaster, “Method for making electrically conductive textiles and textile sensor,” U.S. Pat. No. 10,119,208 B2, Nov. 8, 2018.
[16] ISO 6330:2012—Textiles. Domestic Washing and Drying Procedures for Textile Testing. British Standards Institution: London, U K, 2012.
[17] Erhman A, Heimlich F, Brucken A, et al. Experimental Investigation of the Washing Relaxation of Knitted Fabrics from Polyester Yarn with Stainless Steel Fibres. Fibres Text. East. Eur. 2012; 1(90):90-93.

Although particular embodiments of the invention have been disclosed herein in detail, this has been done by way of example and for the purposes of illustration only. The aforementioned embodiments are not intended to be limiting with respect to the scope of the invention. It is contemplated by the inventors that various substitutions, alterations, and modifications may be made to the invention without departing from the spirit and scope of the invention.

1. A knitted textile comprising:
a knitted textile sensor, wherein the knitted textile sensor comprises an electrically conductive yarn and a plurality of stitches that form a defined sensing stitch pattern, wherein the sensing stitch pattern comprises stitches selected from the group consisting of: jersey stitches; tuck stitches; and/or miss stitches; as well as any combination thereof, and which sensing stitch pattern provides a measurable contact resistance that varies with a force applied to the textile; and
a knitted support structure within which the knitted textile sensor is integrated, wherein the knitted support structure comprises a plurality of stitches that form a defined support stitch pattern, wherein the support stitch pattern comprises stitches selected from the group consisting of: jersey stitches; tuck stitches; and/or miss stitches; as well as any combination thereof, the support stitch pattern having a smaller percentage of tuck stitches than the sensing stitch pattern;
wherein the textile is configured to elongate and retract in the wale direction in a repeatable manner.

2. The knitted textile of claim 1, wherein the support stitch pattern comprises stitches selected from the group consisting of jersey stitches and miss stitches.

3. The knitted textile of claim 1, wherein the support stitch pattern includes one or more double miss stitch arrangements, wherein a double miss stitch arrangement comprises two consecutive miss stitches in the same wale.

4. The knitted textile of claim 1, comprising a boundary zone contiguously interconnecting the courses of the knitted textile sensor and of the knitted support structure, wherein the boundary zone is configured to maintain the position and aspect ratio of the knitted textile sensor relative to the knitted support structure.

5. The knitted textile structure of claim 4, wherein the boundary zone comprises a plurality of stitches that form a defined boundary stitch pattern, wherein the boundary stitch pattern comprises stitches selected from the group consisting of: jersey stitches; tuck stitches; and/or miss stitches; as well as any combination thereof, the boundary stitch pattern having a higher percentage of tuck stitches than the support stitch pattern.

6. The knitted textile structure of claim 5, wherein the boundary stitch pattern has a higher percentage of tuck stitches than the sensing stitch pattern.

7. The knitted textile structure of claim 5, wherein the boundary zone comprises stitches selected from the group consisting of jersey stitches and tuck stitches.

8. The knitted textile structure of claim 4, wherein the boundary zone is at least two wales wide, and wherein the tuck stitches in the boundary zone are staggered.

9. The knitted textile structure of claim 1, comprising a stabilising structure attached to one side of the knitted textile structure, wherein the stabilising structure comprises an elastic fabric.

10. The knitted textile structure of claim 9, wherein the stabilising structure comprises an open fabric configured to enable heat dissipation therethrough.

11. The knitted textile structure of claim 1, wherein 50% of the stitches in the sensing stitch pattern are jersey stitches, and wherein the remaining 50% of stitches comprises a combination of miss stitches and tuck stitches.

12. The knitted textile structure of claim 11, wherein more than 25% of the stitches in the sensing stitch pattern are tuck stitches, and the remainder of the stitches in the sensing stitch pattern are miss stitches.

13. The knitted textile structure of claim 1, wherein the electrically conductive yarn comprises a multifilament yarn.

14. The knitted textile structure of claim 1, wherein the structure is arranged to form a sleeve.

15. A garment comprising the knitted textile structure of claim 1.

16. The garment of claim 15, wherein the garment is a sleeve.

17. A knitted textile sensor comprising:
a support region comprising yarn and a plurality of stitches that form a defined support stitch pattern wherein the support stitch pattern comprises stitches selected from the group consisting of: jersey stitches; tuck stitches; and/or miss stitches; and wherein at least 50% of the stitches in the support stitch pattern are jersey stitches; and
a sensor region comprising an electrically conductive yarn and a plurality of stitches that form a defined sensing stitch pattern, wherein the sensing stitch pattern comprises stitches selected from the group consisting of: jersey stitches; tuck stitches; and/or miss stitches; wherein 50% of the stitches in the sensing stitch pattern are jersey stitches, at least 25% of the stitches in the stitch pattern are tuck stitches, and the remainder of the stitches in the sensing stitch pattern are miss stitches; and wherein the stiffness of the sensing stitch pattern is higher than the stiffness of the support stitch pattern.
